(19) United States
(12) Patent Application Publication
(10) Pub. No.: US 2026/0069565 A1
(43) Pub. Date: Mar. 12, 2026

(54) Method of Treating Amyotrophic Lateral Sclerosis

(71) Applicants: Provectus Pharmatech, Inc., Knoxville, TN (US); UTI Limited Partnership, Calgary (CA)

(72) Inventors: Aru NARENDRAN, Calgary (CA); Yusheng Zhao, Calgary (CA); Dominic Rodrigues, Knoxville, TN (US); Edward V. Pershing, Knoxville, TN (US); Eric A. Wachter, Oak Ridge, TN (US)

(73) Assignees: Provectus Pharmatech, Inc., Knoxville, TN (US); UTI Limited Partnership, Calgary (CA)

(21) Appl. No.: 19/323,435

(22) Filed: Sep. 9, 2025

Related U.S. Application Data
(60) Provisional application No. 63/693,919, filed on Sep. 12, 2024.

(51) Int. Cl. A61K 31/352

(52) U.S. Cl. CPC A61K 31/352

(57) ABSTRACT

The present invention is directed to a pharmaceutical composition and method for treating a subject diagnosed amyotrophic lateral sclerosis (ALS) with a pharmaceutical composition containing dissolved or dispersed therein a SOD1 and/or TDP-43 aggregation-inhibiting amount of a rose bengal (RB) compound that is a pharmaceutically acceptable salt of RB, RB lactone, a RB amide, an aromatic RB derivative, wherein the aromatic derivative is an ester or amide formed from an alcohol or monosubstituted amine having a 5- or 6-membered aromatic ring, or a 5,6- or 6,6-fused aromatic ring system that contains 0, 1, or 2 hetero ring atoms that are independently nitrogen, oxygen or sulfur. This treatment method is typically repeated a plurality of times or until the subject no longer needs it.

### CROSS-REFERENCE TO RELATED APPLICATION

This application claims priority to U.S. application Ser. No. 63/693,919, filed on Sep. 12, 2024, whose disclosures are incorporated herein by reference.

### BACKGROUND ART

Amyotrophic lateral sclerosis (ALS), also referred to as Lou Gehrig's disease, is a fatal progressive neurodegenerative disorder for which approximately 80% of cases die within 2 to 5 years of diagnosis. It is estimated that more than 200,000 people are living with ALS worldwide, and that the number is predicted to increase by 70% by 2040. There is no present cure for ALS, and current treatment only provides symptomatic relief.

The cause of ALS is largely unknown. Symptoms begin with weakness and muscle twitches, and then spread to involve most muscles. ALS causes the loss of nerve cells in the spinal cord and brain.

The accumulation of two proteins in neural tissues, such as the brain and spinal cord, correlates with the progression of ALS. One of those proteins is the metalloenzyme superoxide dismutase 1 (SOD1) (E.C. No. 1.15.1.1). The second of these two proteins is transactive response DNA binding protein, also known in the art more colloquially as TAR DNA-binding protein 43 or TDP-43.

Misfolded SOD1 (sometimes also referred to as CuZnSOD because it binds to copper and zinc) was the first protein linked to ALS [Hnath et al., Biophys J (Jun. 7, 2022) 121:2084-2095]. SOD1 alternately catalyzes the [symbol] (or partitioning) of the [symbol] (O⁻₂) anion [symbol] into normal molecular [symbol] (O₂) and [symbol] [symbol] (H₂O₂). Superoxide is produced as a by-product of oxygen metabolism and, if not regulated, can cause many types of cell damage.

SOD1 is a 32 kDa homodimer that is one of three SOD1s present in humans and is found in the cytoplasm. The two subunits are tightly joined back-to-back via a single disulfide bond as well as hydrophobic and some electrostatic interactions. The ligands of copper and zinc are six [symbol] and one [symbol] side-chains; one histidine is bound between the two metals [Tainer et al., Nature (1983) 306 (5940): 284-287].

SOD1 has been implicated in apoptosis, familial ALS, and Parkinson's disease. Mutations in SOD1 are associated with about 20% of familial ALS (fALS). The wild-type enzyme has also been implicated in a significant fraction of sporadic ALS (sALS) cases, which represent 90% of ALS patients [Gagliardi et al., Neurobiol Dis (2010 August) 39 (2): 198-203].

Recent studies have discovered misfolded SOD1 in sALS patient tissue samples without any genetic mutations and post-translational modifications to SOD1 that either occur naturally or from environmental toxins, have been shown to destabilize SOD1 leading to aggregation. Mutations, loss of metals, crowding, and post-translational modifications to SOD1 lead to destabilization of the dimer interface. De-metalated SOD1 monomers are highly prone to aggregation, and the large, insoluble inclusion bodies they form have been studied since the protein was linked to ALS.

More than 170 different mutations in superoxide dismutase 1 (SOD1), are found in familial cases of ALS. Most of those SOD1 mutants show little change in enzymatic function, suggesting that toxicity derives not from a loss of native function but from a gain of toxic function [Sangwan et al., Proc Natl Acad Sci, USA (2017) 114 (33): 8770-8775].

Saeed et al., Neurology (2009) 72:1634-1639 reported that the alanine to valine mutation at codon 4 (A4V) of SOD1 causes a rapidly progressive dominant form of ALS with exclusively lower motor neuron disease and is responsible for 50% of SOD1 mutations associated with fALS in North America. This A4V mutation is rare in Europe.

Sangwan et al., above, identified residues 28-38 of SOD1 as having the potential to form a toxic amyloid oligomer based on mutational studies of others and their own work. They engineered a single-residue substitution, P28K, to increase solubility. That engineered polypeptide formed crystals; they analyzed the crystal structure and found that the crystal structure showed a twisted β-sheet built of antiparallel, out-of-register β-strands. Those authors, and others since, refer to that structure as the “corkscrew.”

Each β-strand in the sheet contains eight residues from Lys28 to Ile35. The three C-terminal residues, Lys36, Gly37, and Leu38, adopt a β-hairpin conformation, positioning the C-terminal carboxylate to hydrogen-bond with the N-terminal residue of an adjacent strand. The twist of the sheet is left-handed, as is commonly observed for β-sheets, and undergoes a full turn every 16 strands [Sangwan et al., Proc Natl Acad Sci, USA (2017) 114 (33): 8770-8775].

Sangwan et al. reported that the corkscrew architecture differs markedly from amyloid fibrils in that sheets from adjacent corkscrews do not mate together tightly as sheets do in amyloid fibrils, but instead contact weakly through polar and charged side chains scattered over the exterior of the corkscrew and water-mediated contacts. Consequently, unlike amyloid fibrils, the corkscrew has no dry interface between sheets to stabilize its assembly. Rather, the corkscrew assembly is stabilized by weaker hydrophobic forces arising from the concave interior filled with aliphatic side chains of Val29, Val31, Ile35, and Leu38. After further studies, the authors concluded that their data suggested segment 28-30 is both necessary and sufficient for motor neuron toxicity.

Proteinaceous aggregates of SOD1 found in ALS patients are correlated to ALS progression. SOD1 normally functions in homodimer form. The SOD1 dimer can dissociate into monomers, lose their metal ions, and are more prone to aggregation. The monomers interact to form oligomers, and higher-order species, and eventually aggregated fibrils. In this mechanism, the formation of the trimer is a necessary step in the formation of larger insoluble fibrils. This so-called “on pathway” synthetic activity is schematically illustrated in FIG. 2, which is taken from Hnath et al., Biophys J (Jun. 7, 2022) 121:2084-2095.

Many models for the aggregation of proteins focus on nucleation and growth processes, but they often assume that aggregation is only a linear process (i.e., on-pathway), similar to the crystallization processes. Validation of the models is primarily based on data from thioflavin T (ThT) aggregation studies. ThT only binds across β-sheets in amyloid fibrils. Thus, this method cannot account for protein conformations that have low percentages of β-sheets as, for example, in SOD1 trimers [Hnath et al., Biophys J (Jun. 7, 2022) 121:2084-2095].

Present theories regarding other diseases involving deposition of intracellular fibrils such as Alzheimer's disease and Parkinson's disease in which amyloid and synuclein fibrils, respectively, are thought the principal toxic species responsible for neurodegenerative diseases and remain diagnostic and therapeutic targets. More recent work in this area indicate that smaller, soluble molecules such as amyloid oligomers are the toxic entities. Similarly, non-native SOD1 trimers are toxic to motor neuron-like cells, whereas larger aggregates are protective.

Hirota and Hall [Hall et al., FEBS Lett (2015) 589:672-679; and Hirota et al., Biophys Rev (2019) 11:191-208] developed a model for protein aggregation that removes the assumption that aggregation is a linear process. Those workers also include changes in concentrations over time as an output for their model, instead of equating the system to ThT curves. They show that, during protein aggregation where the intermediate is on-pathway, the concentration of the intermediate has a positive correlation with the concentration of larger aggregates. The opposite occurs when the intermediate is off-pathway and competing with larger aggregate formation; in an off-pathway case, there is a strong negative correlation between intermediate and larger aggregates [Hnath et al., Biophys J (Jun. 7, 2022) 121:2084-2095].

The Hnath et al. group posits that protein aggregation is a protective mechanism against increased population of misfolded proteins. They provide evidence and argue that production of trimeric SOD1 is an “off-pathway” in the formation of larger, insoluble protective SOD1 inclusion bodies such that the formation of the toxic soluble trimer directly competes with insoluble fibril formation. This alternative synthesis of fibrils is shown schematically in FIG. 3, which is also taken from Hnath et al., Biophys J (Jun. 7, 2022) 121:2084-2095.

Those workers further contend the finding that SOD1 trimers are formed off-pathway suggests two possible therapeutic intervention strategies: 1) inhibit native dimer dissociation, which results in loss of metals and mis-folding; and/or 2) promote SOD1 aggregation, thereby depleting the formation of SOD1 trimers [Hnath et al., Biophys J (Jun. 7, 2022) 121:2084-2095].

As there is currently no cure or disease-modifying treatment for ALS, for most people with ALS, the main treatment includes managing symptoms (i.e., symptomatic therapy). JingSi et al., Front Pharmacol (2022) 13:1054006 note that 53 new drugs were evaluated in clinical trials from 2020 to 2022. Those authors also narrowed their discussion to the three drugs approved for treating ALS by the U.S. Food and Drug Administration (FDA): riluzole, edaravone, and Relyvrio™ (AMX0035). These medications are said to improve short-term survival (i.e., by about 3 month) and/or slow down physical decline.

Riluzole is an anti-glutamate agent that inhibits the overexcitation of motor neurons by inhibiting presynaptic glutamate release. Edaravone is a free radical scavenger that reduces oxidative stress. Its clinical results have been inconclusive, inconsistent, and contradictory, possibly due to genetic heterogeneity. Relyvrio™ inhibits apoptosis, reducing neuronal death by simultaneously targeting the mitochondria and endoplasmic reticulum. Its approval was due to disease severity and the need for safe treatment.

According to JingSi et al. above, clinical trials of ALS treatments fall generally into eight mechanistic groups: anti-apoptotic; anti-inflammatory; anti-excitotoxicity; regulated integrated stress response; neurotropic factors and neuroprotection; gene therapy; anti-aggregation; and others. Of those eight groups, gene therapy and anti-aggregation are the only disease-modification strategies.

TDP-43 is the main component of the intraneuronal inclusion bodies found in ALS patients. TDP-43 accumulation is the characteristic feature in more than 95% of all ALS cases and about 50% of fALS cases. The full three-dimensional (3D) atomic structure of TDP-43 is not yet resolved; however, the major domains of TDP-43 have been solved, including N-terminal domain (NTD), RNA recognition motifs 1/2 (RRM1/2), and C-terminal domain (CTD). All these regions play partial roles in its pathological signaling pathways and amyloidogenic aggregation.

TDP-43 is a ubiquitous protein that is encoded by the TARDBP gene and belongs to the heterogeneous nuclear ribonucleoprotein (hnRNP) family. In normal cells, TDP-43 is mainly present in the nucleus and plays important roles in RNA regulation, such as transcriptional regulation, alternative splicing, and mRNA stabilization. Under pathological conditions, cleavage, hyperphosphorylation, and ubiquitination of TDP-43 can occur.

These post-translational modifications lead to cytoplasmic accumulation and aggregation of TDP-43. In particular, phosphorylation of TDP-43 at serine 403/404 and 409/410 (p-TDP-43) can result in the pathological inclusions observed in TDP-43 proteinopathies [Jo et al., Exp Mol Med (2020) 52:1652-1662.

ALS pathology mainly affects both upper motor neurons (UMN) in the cortex and lower motor neurons (LMN) in the brainstem and spinal cord. However, ALS can also affect neighboring cell populations, such as glial cells, peripheral inflammatory cells, and muscles, as ALS is increasingly considered a multi-systemic disease that culminates in motor neuron death. For example, astrocytes and microglia have been implicated in the release of pro-inflammatory mediators that lead to chronic neuroinflammation and motor neuron toxicity. In addition, the selective overexpression of mutant SOD1 in skeletal muscle was shown to cause mitochondrial abnormalities, induce microglial activation in the central nervous system (CNS), and result in severe muscle atrophy in mice [Le Gall et al., J. Pers. Med. 2020, 10:101, 34 pages].

The present invention relates to disease-modifying strategies, and particularly to anti-aggregation strategies. That mechanistic group includes anti-TDP-43 aggregation, anti-SOD1 aggregation, and antibody or autophagy enhancement.

As discussed in further detail hereinafter, the present invention contemplates treatment of ALS by inhibiting the formation of the insoluble aggregates of one or both of TDP-43 and SOD1. That inhibition of aggregate formation is accomplished by 1) blocking the binding of RNA to the RRM1/2 interface and thereby restoring the normal RNA, metabolism; 2) interacting with the CTD region of TDP-43, which is the main aggregation-prone region of the molecule; 3) stabilizing the SOD1 dimer to prevent its monomerization; and 4) inhibit aggregation of wild type (WT) and mutant SOD1 by interacting with the oligomers.

### BRIEF SUMMARY OF THE INVENTION

In one aspect, the present invention is directed to a method and to a pharmaceutical composition for treating a subject with diagnosed amyotrophic lateral sclerosis (ALS) with a pharmaceutical composition containing dissolved or dispersed therein a SOD1 and/or TDP-43 aggregation-inhibiting amount of a rose bengal (RB) compound that is a pharmaceutically acceptable salt of RB, rose bengal lactone, a RB amide whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic RB derivative, wherein the aromatic derivative is an ester or amide formed from an alcohol or monosubstituted amine having a 5- or 6-membered aromatic ring, or a 5,6- or 6,6-fused aromatic ring system that contains 0, 1, or 2 hetero ring atoms that are independently nitrogen, oxygen or sulfur.

In another aspect of the invention, the treatment method is repeated as prescribed a plurality of times over the following months or years or until the subject is no longer in need.

In another aspect of the invention, the aromatic ring substituent of the pharmaceutical composition is selected from one or more of the group consisting of one or more of

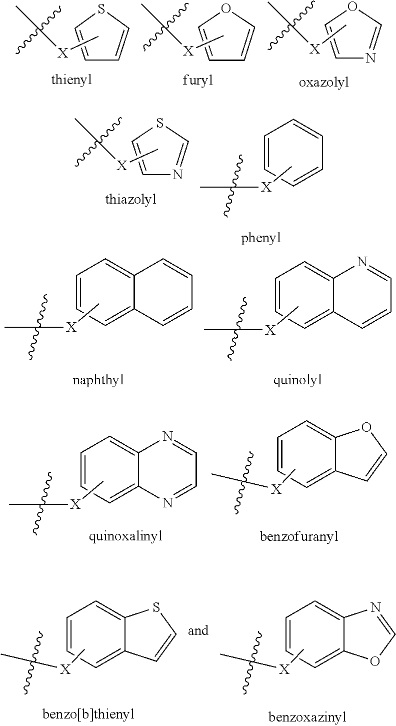

where

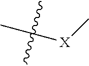

is

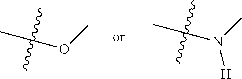

providing an ester, amide (—NH₂) or a monosubstituted amide, respectively.

In another aspect of the invention, RB compound of the pharmaceutical composition is a C₁-C₄ alkyl ester or rose bengal disodium salt.

In another aspect of the invention, RB compound of the pharmaceutical composition is rose bengal disodium salt.

In another aspect of the invention, the pharmaceutical composition is a liquid at room temperature.

In another aspect of the invention, the liquid pharmaceutical composition is an aqueous composition containing at least about 90% w/v water.

In another aspect of the invention, the aqueous liquid pharmaceutical composition has an osmolality of about 300 to about 500 mOsm/kg.

In another aspect of the invention, the pharmaceutical composition is a solid at room temperature.

In another aspect of the invention, the pharmaceutical composition contains a SOD1 aggregation-inhibiting amount of said RB compound.

In another aspect of the invention, the pharmaceutical composition contains a TDP-43 aggregation-inhibiting amount of said RB compound.

In another aspect of the invention, the method and pharmaceutical composition is for treating a human subject with diagnosed amyotrophic lateral sclerosis (ALS).

### BRIEF DESCRIPTION OF THE DRAWINGS

In the drawings forming a portion of this disclosure:

FIG. 1, in two parts as FIG. 1A and FIG. 1B, is a line diagram of the primary sequence of the 43 kD human full-length wild-type TDP-43, illustrating the N-terminal domain (NTD), RNA recognition motifs 1/2 (RRM1/2) and the C-terminal (CTD) domains FIG. 1A), and ribbon cartoons of the solved three-dimensional (3D) structures of NTD, RRM1/2, and CTD. These figures are adapted from Rao et al., Eur J Med Chem (2021 Dec. 5) 225:113753;

FIG. 2 is a depiction of the on-pathway formation of the SOD1 trimer as a necessary step in the formation of larger insoluble fibrils using ribbon cartoons of solved 3D structures of the dimer, and is adapted from Hnath et al., Biophys J. (2022 Jun. 7) 121 (11): 2084-2095;

FIG. 3, also adapted from the above Hnath et al. publication, illustrates an off-pathway formation of the toxic soluble trimer that directly competes with insoluble fibril formation;

FIG. 4 is a computer-prepared model of the TDP-43 RRM1/RRM2 interface to which a molecule of RB is docked, wherein the RB molecule is illustrated as the black ball and stick construct and the background shows the surface of that interface. This model and those discussed hereinafter were prepared using the AutoDock Vina [Dr. Oleg Trott, Molecular Graphics Lab, Scripps Research Institute, LaJolla, CA] and BIOVIA Discovery Studio [Dassault Systèmes BIOVIA, Discovery Studio Modeling Environment, Release 2017, San Diego, CA] platforms to perform in silico flexible ligand-receptor docking and determine overall binding energy based on inter-atomic distances;

FIG. 5 is a depiction of the RB binding of FIG. 4 in which salient amino acids and their positions in the TDP-43 protein sequence on or near the interface surface are noted using “A” for the single chain of the protein to which the RB is docked, the residue involved in docking by the amino acid three-letter code, and the position number of that involved residue in the protein sequence, with dashes and lower case letters indicative of the type of bonding predicted between atoms or region of RB and the recited amino acid residues, where “a” indicates a hydrogen bond, “b” indicates hydrophobic interaction, and “c” and “d” noted in later figures depict electrostatic and halogen bond interactions, respectively;

FIG. 6 is a computer-prepared model, as previously described, of the best binding pose of RB with TDP-43 CTD portion, wherein the RB molecule is illustrated as the black stick construct atop portions of two chains of the CTD;

FIG. 7 is a depiction of the best RB binding pose of FIG. 6 in which salient amino acids and their positions in the TDP-43 CTD protein sequence on or near the site of that pose, and that here two chains (A and B) are involved in docking as is shown in FIG. 6;

FIG. 8 is a computer-prepared model, as previously described, of the interface between two wild-type SOD1 monomers in a dimer molecule to which a molecule of RB is docked (noted by the arrow point), wherein the RB molecule is illustrated as the black ball and stick construct and the background shows the surface of that interface, and where the relative hydrophobicity of the dimer surface is shown by the white to black hydrophobicity scale shown at the lower left of the figure;

FIG. 9 is an enlarged copy of the bound RB molecule in the cleft between the two monomers of the SOD1 dimer;

FIG. 10 is a depiction of the region containing the bound RB and the amino acid residues with which the RB can dock and other residues in the vicinity of that binding surface, and in which A and H, two chains of the dimer that are involved in the best docking pose;

FIG. 11 is a computer-prepared model, as previously described, of the best docking pose of an RB molecule with SOD1 28-38 corkscrew oligomer model in which the RB is illustrated as the black construct atop of the monomer chain strands;

FIG. 12 is an enlarged view of upper right portion of FIG. 11, in which the two gray, tryptophan ring systems interact with black RB molecule; and

FIG. 13 is a depiction of the region of FIGS. 11 and 12 containing the bound RB and the SOD1 28-38 corkscrew oligomer amino acid residues with which the RB interacts, here with three chains, A, B and C, and other residues in the vicinity of that binding surface.

### MOLECULAR DOCKING

There is no well-defined binding pocket associated with amyloidogenic protein aggregation. Therefore, a blind docking strategy is used here.

In this in silico method, nine possible docking poses are generated by default by the computer program and ranked according to binding affinity. Binding affinity is determined by the calculated energy in kcal/mol. The lower energy the system has, the more stable the drug-protein complex is, the higher binding affinity. Non-bonded intermolecular interactions can be monitored to explore the binding mechanism in detail, including electrostatic interactions, hydrogen bonding, and hydrophobic interactions.

The appropriateness of the results obtained through these studies can be and were corroborated by comparisons based on high-affinity ligands reported by the literature and ligand in complex with protein targets obtained by X-ray diffraction, solid NMR, solution NMR, and the like.

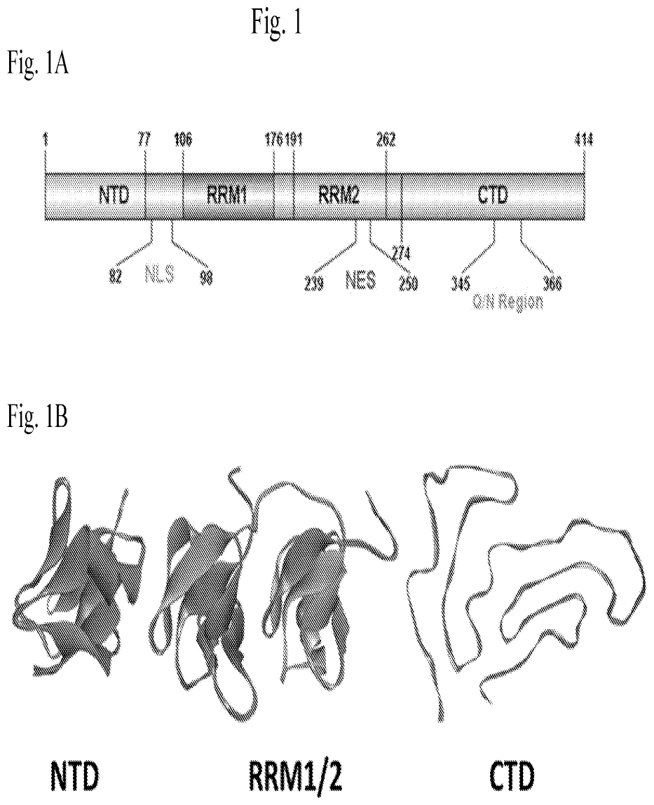

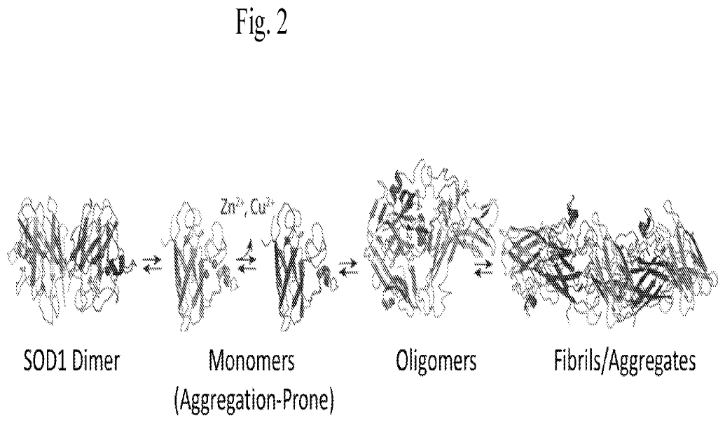

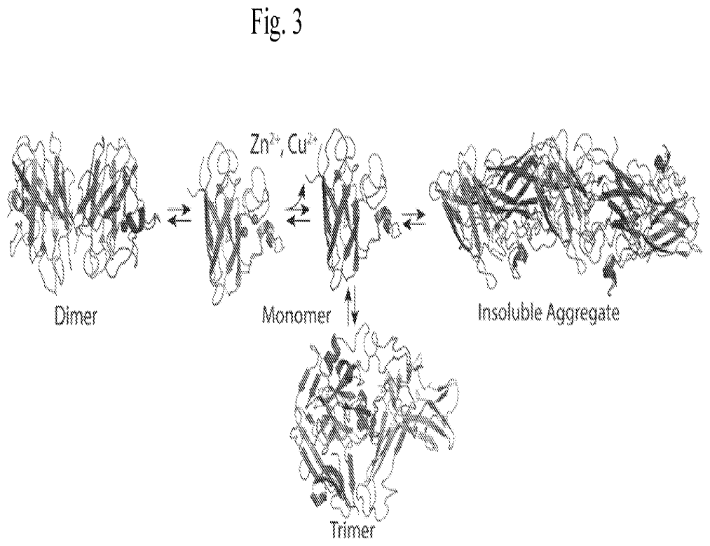

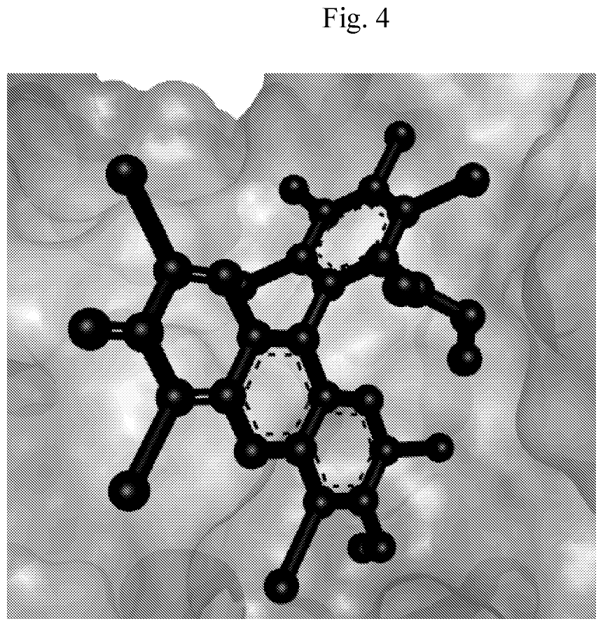

Best docking poses were determined using the AutoDock Vina [Dr. Oleg Trott, Molecular Graphics Lab, Scripps Research Institute, LaJolla, CA] and BIOVIA Discovery Studio [Dassault Systèmes BIOVIA, Discovery Studio Modeling Environment, Release 2017, San Diego, CA] platforms to perform in silico flexible ligand-receptor docking and determine overall binding energy based on inter-atomic distances as discussed above. As can be seen from FIGS. 1B, 2, 3, 6, 11, and 12 using ribbon modeling, and from FIGS. 4, 8, and 9 using space-filling modeling for the respective protein portions.

Further information regarding these docking pose systems and their use can be found in Trott et al., J Comput Chem (2010) 31:455-461, 2010; Eberhardt et al., J Chem Inf Model (2021) 61:3891-3898; and Rao et al., Eur J Med Chem (2021 Dec. 5) 225:113753.

### DETAILED DESCRIPTION OF PREFERRED EMBODIMENTS

The present invention is directed to treating a subject diagnosed amyotrophic lateral sclerosis (ALS) with a pharmaceutical composition containing dissolved or dispersed therein a SOD1 and/or TDP-43 aggregation-inhibiting amount of a rose bengal (RB) compound that is a pharmaceutically acceptable salt of RB, rose bengal lactone, a RB amide whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic RB derivative, wherein the aromatic derivative is an ester or amide formed from an alcohol or monosubstituted amine having a 5- or 6-membered aromatic ring, or a 5,6- or 6,6-fused aromatic ring system that contains 0, 1, or 2 hetero ring atoms that are independently nitrogen, oxygen or sulfur. The present invention is directed to this pharmaceutical composition and method of treatment of a subject diagnosed with ALS with this pharmaceutical composition. A human is typically the subject diagnosed with ALS and in need of treatment and to whom a contemplated pharmaceutical composition and method is applied. It is also contemplated that other mammals could be the subject for the present invention. This treatment method is repeated as prescribed a plurality of times over the following months or years or until the subject no longer needs it.

A contemplated rose bengal molecule of an embodiment of the present invention has a structural formula shown in Formula I, below, where X is oxygen or nitrogen, “n” is zero or one. When X is oxygen, n is zero and absent so that the RB compound is: a) rose bengal where —X—R¹ is —O—H; b) a pharmaceutically acceptable salt of RB where —X—R¹ is O M⁺ and where M⁺ is a pharmaceutically acceptable cation, including a proton (H⁺); c) a C₁-C₄ alkyl ester; or d) an aromatic ester as defined below. Alternatively, when X is a nitrogen atom, n is 1, and R² is present along with R¹. As such, R¹ and R² can be the same or different, and C(O)—NR¹R² is an amide whose nitrogen atom is a) unsubstituted [—X—(R¹R²) and both R¹ and R² are hydrogen (H)]; is b) substituted with one or two C₁-C₄ alkyl groups, or together with the amido nitrogen atom R¹ and R² form a 5- or 6-membered ring; or is c) an aromatic amide that is preferably monosubstituted in that R¹ is hydrogen and R² is the aromatic substituent discussed below.

For ease of description, an aromatic ester or aromatic amide are collectively referred to as an aromatic derivative. As such, those derivatives are formed from an alcohol or amine, preferably monosubstituted, having a single 5- or 6-membered aromatic ring or a 5,6- or 6,6-fused aromatic ring system that contains 0, 1, or 2 hetero ring atoms that are independently nitrogen, oxygen or sulfur.

Illustrative examples of such aromatic alcohol ester portions are shown and named below, where O is an oxygen atom and line-O indicates the ring-oxygen can be from any available carbon of the ring, and the O-line crossed by a wavy line indicates that the depicted alkoxy group is also a portion of another molecule, the esterified RB molecule. Where X is a nitrogen atom, analogous amide structures are contemplated.

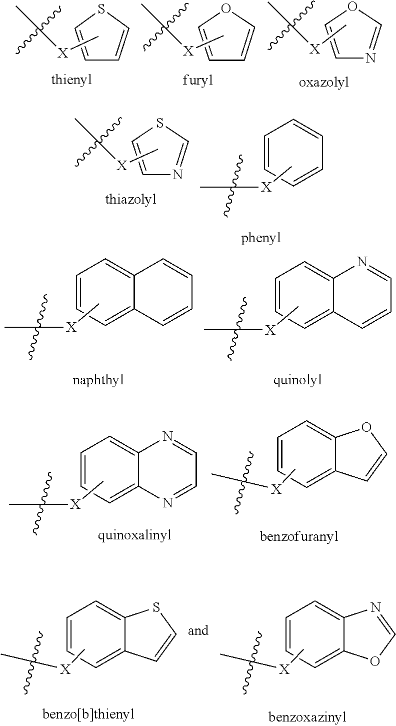

where

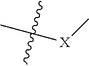

is

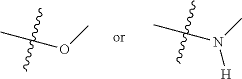

providing an ester, amide (—NH₂) or a monosubstituted amide, respectively.

Rose bengal (RB) itself is a preferred RB compound, and its disodium salt, rose bengal disodium (RBD), is the most preferred RB compound. The World Health Organization (WHO) now refers to this same material as rose bengal sodium. These compounds are used illustratively herein for the group of RB compounds.

The chemical name for rose bengal is 4,5,6,7-tetrachloro-2′, 4′,5′,7′-tetraiodo-fluorescein. A preferred form, rose bengal disodium (RBD), has the following structural formula:

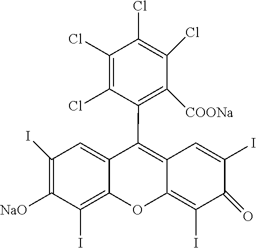

Certain details of this preferred embodiment for a contemplated composition are described in U.S. Pat. Nos. 5,998,597, 6,331,286, 6,493,570, and 8,974,363, whose disclosures are incorporated by reference herein in their entireties. The above patents describe the use of RBD to kill cancer cells.

### Docking Pose Results

The results of the docketing pose studies indicate that RB can bind to various positions of both SOD1 and TDP-43.

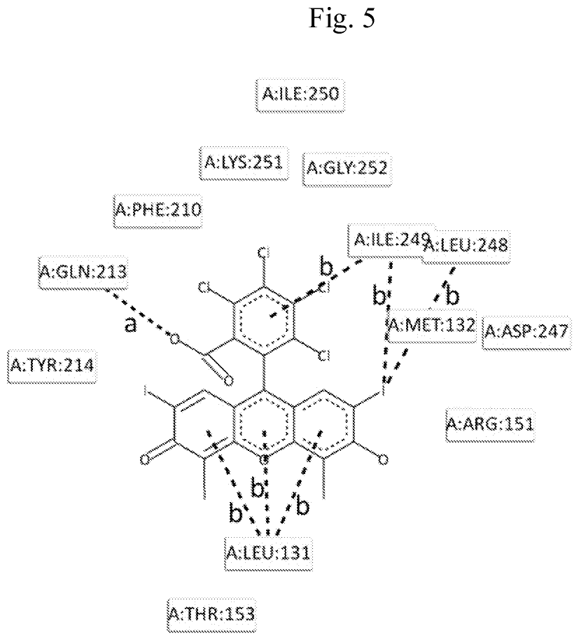

More specifically, RB as RBD had a best binding pose at the RRM1/RRM2 interface of the TDP-43 dimer. That binding is illustrated in FIG. 4 using computer-generated three-dimensional (3D) model with the ball and stick model of RBD and in the two-dimensional (2D) depiction of FIG. 5 showing the hydrophobic bonding of the tricyclic ring system to the leucine at position 131 and the isoleucine, leucine and methionine of positions 249, 248, and 132, respectively. The RBD aromatic ring carboxyl group is shown hydrogen-bonded to the glutamine at position 213. The single bond between the RB single and triple ring systems permits rotation of the single ring to fit into the binding pocket groove. The binding affinity was calculated to be −5.8 kcal/mol.

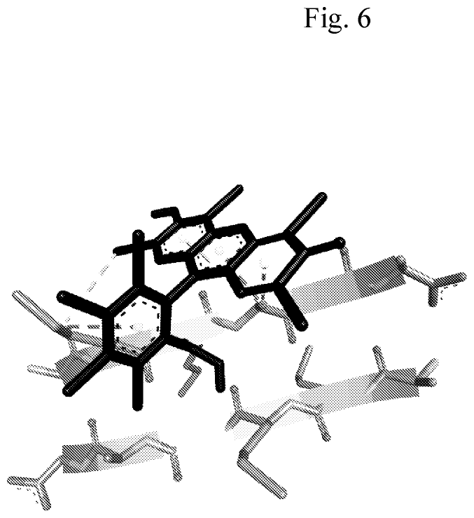

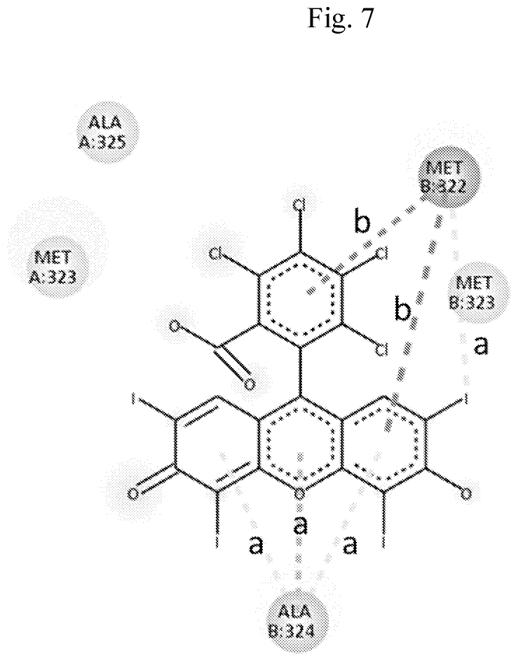

FIGS. 6 and 7 illustrate the best docking pose for RBD with the TDP-43 C-terminal domain (CTD). FIG. 6 illustrates the RBD as a ball and stick model interacting with a ribbon model of two strands, whereas FIG. 7 illustrates the more specific calculated interactions as discussed below.

The calculated binding affinity for the interactions for RBD shown in FIGS. 6 and 7 amounted to −4.6 kcal/mol. The position of the binding pose indicates that the CTD steric zipper assembly is blocked. That blockage occurs due to hydrophobic interactions (a) between the tricyclic ring and alanine 324, as well as between a tricyclic iodine and methionine 322 of a second strand (B) and electrostatic interactions (pi-sulfur) between both aromatic ring systems and methionine 322 of that same second chain (B).

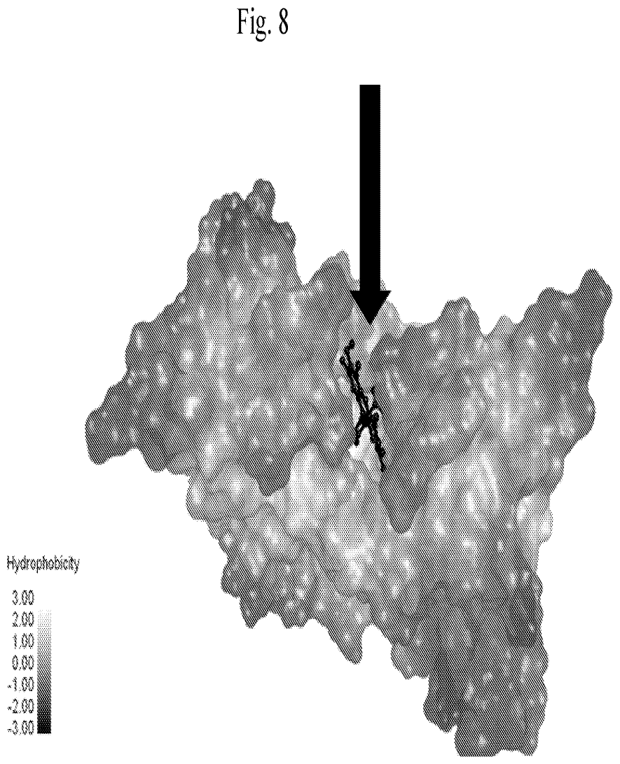

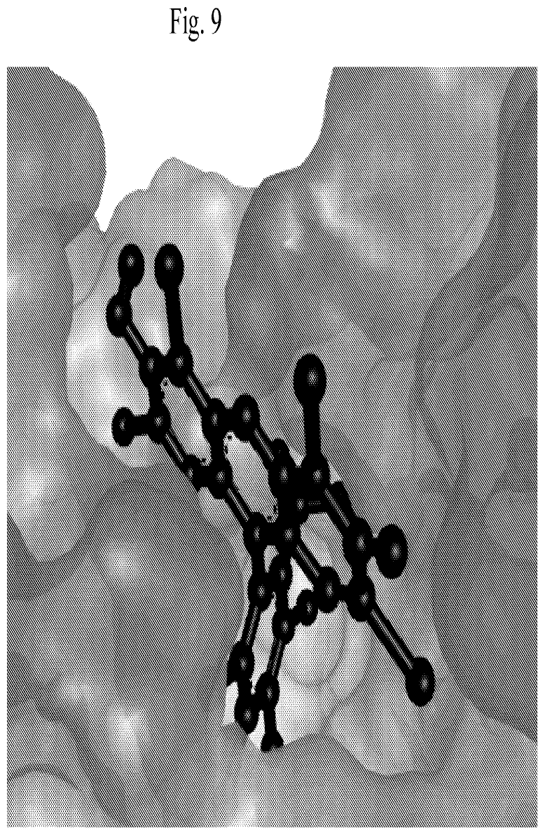

FIGS. 8, 9, and 10 illustrate the best docking pose of RBD between the monomers of a SOD1 dimer. FIGS. 8 and 9 illustrate two versions of the same pose, with FIG. 8 being a “long shot” and FIG. 9 being a “close-up.” As can be seen best in FIG. 9, RBD fits well into the deep groove of the SOD1 dimer hydrophobic binding pocket. A binding affinity of −7.2 kcal/mol was calculated for that interaction.

The aromatic carboxyl-containing ring goes deep into the groove to occupy the space. The tricyclic ring is confined into the pocket by the electrostatic interactions with lysine 9 that is facilitated by several hydrophobic interactions and hydrogen bond interactions.

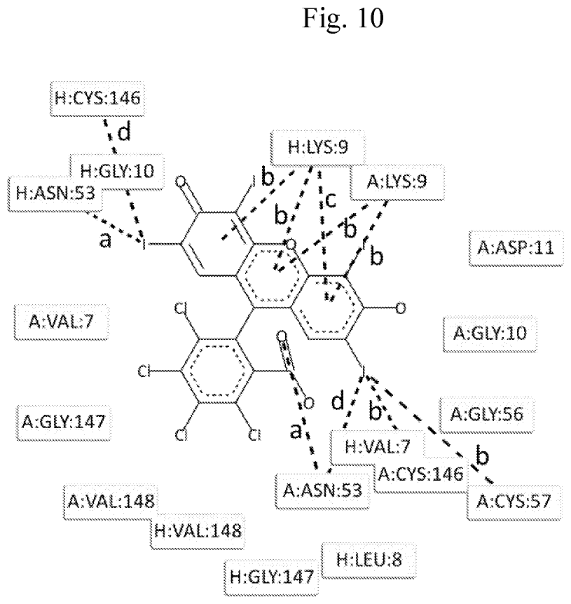

Examining FIG. 10, it is seen that hydrogen bonds are formed between the RBD tetrachorophenyl-carboxyl group and the asparagine 53 of chain A and between a tricyclic iodine and asparagine 53 of chain H. Hydrophobic interactions are shown between another tricyclic iodine and valine 7 of the H chain and a cysteine 57 of the A chain. Similar hydrophobic interactions are shown between the tricyclic ring and lysines 9 and 9 of each of the H and A chains. An electrostatic interaction is also shown between the tricyclic rings and lysine 9 of the H chain. A halogen bond is also shown between the first-noted tricyclic iodine cysteine 146 of the H chain.

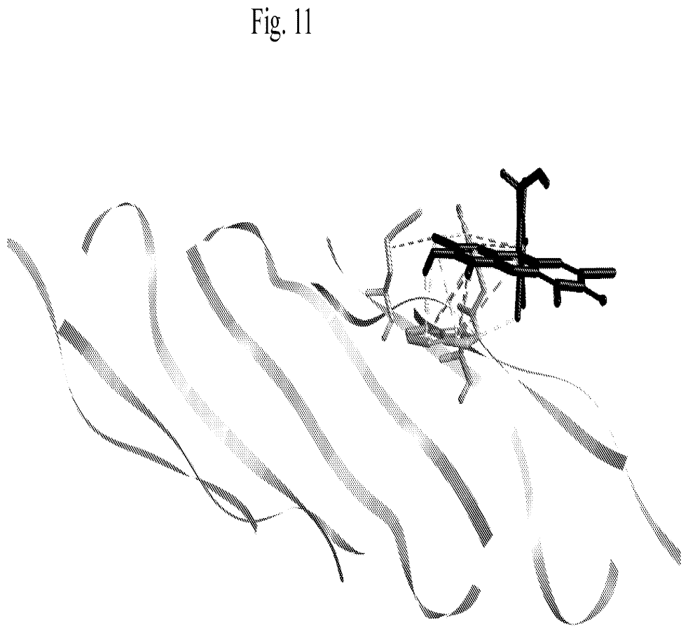

FIGS. 11, 12, and 13 illustrate a best docking pose for RBD with three of eight strands of the SOD1 28-38 corkscrew model of Sangwan et al., Proc Natl Acad Sci, USA (2017) 114 (33): 8770-8775. The depicted binding affinity was-6.1 kcal/mol.

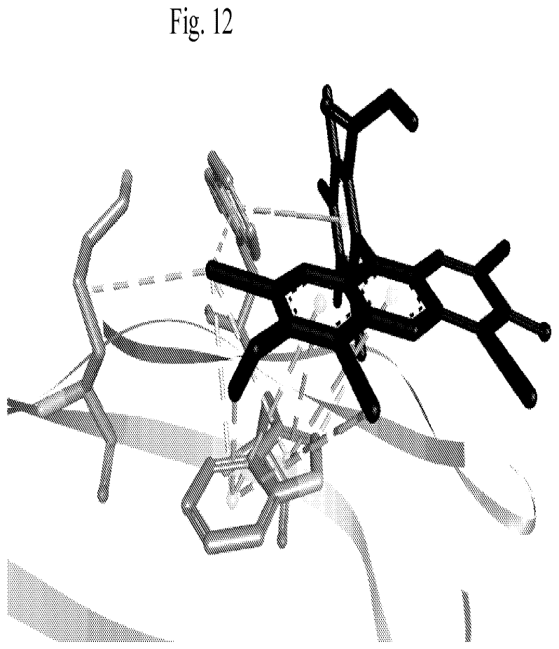

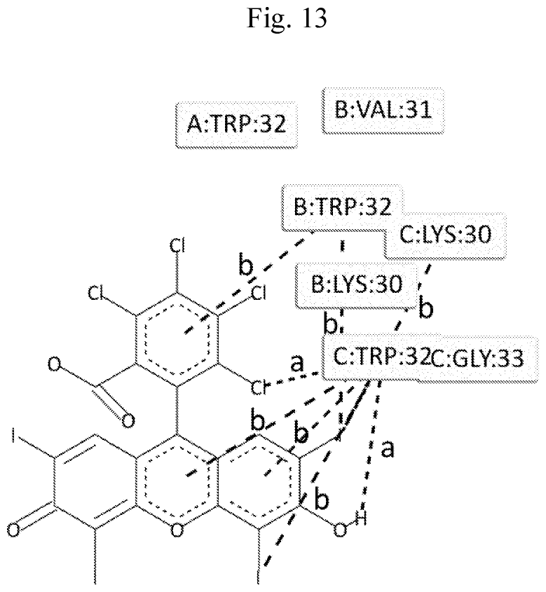

FIGS. 11 and 12 illustrate “long shot” and “close-up” views, respectively, of a RBD best docking pose in which the aromatic tricyclic rings interact hydrophobically with tryptophan 32 and lysine 30 of strand C, and the RBD aromatic carboxyl interacts with tryptophan 32 of strand B via hydrophobic interaction. A hydrogen bond is also shown between the phenolic hydroxyl and tryptophan 32 of strand C. The conformation of RBD, facilitated by C—C single bond, enables interaction with 2 adjacent strands effectively. FIG. 13 shows a 2D depiction of those interactions.

The table below summarizes the above binding affinities. Similar study carried out with the SOD1 A4V mutant was also carried out and its binding affinity is included in the table below.

| | | RB BINDING |
|---|---|---|
| BINDING | | AFFINITY |
| PARTNER | DOMAIN | (kcal/mol) |
| TDP-43 | RRM1/2 Interface | −5.8 |
| TDP-43 | C-Terminal Domain (CTD) | −4.6 |
| SOD1 | OLIGOMER | −6.1 |
| SOD1 | DIMER INTERFACE | −7.2 |
| SOD1 | SOD1-A4V DIMER INTERFACE | −6.8 |

### Subjects and Pharmaceutical Compositions

A human is typically the subject diagnosed with ALS and in need of treatment and to whom a contemplated pharmaceutical composition containing RB, RBD, another pharmaceutically acceptable salt or a rose bengal compound is administered. A genetically-engineered mouse is also a contemplated subject recipient. In vitro cultured affected cells of an ALS patient or genetically-engineered mouse are also possible subjects of such a treatment.

Illustrative of the many useful genetically-engineered mice include those referred to literature as G93A SOD-1 mice that have provided insights into the disease's pathology, revealing a correlation between mutant SOD expression and motor neuron degeneration. These mice are commercially available from Naason Science, Inc., 123 Saengmyung-Ro, Osong-eup, Korea.

rNLS8 (NEFH-hTDP-43-ΔNLS) double transgenic ALS mice are generated by breeding mice having the NEFH-tTA transgene with mice having the tetO-hTDP-43-ΔNLS transgene. This TARDBP model was originally developed and reported by Walker et al. Acta. Neuropathol., 130:643-670, 2015. These mice can also be used as a TDP-43 pathology model of frontotemporal dementia (FTD) or frontotemporal lobar degeneration (FTLD). This mouse strain is available from Biospective, Inc., 1255 Peel, Suite 560 Montréal, Quebec H3B 2T9, Canada. Both SOD1 G93A mice and rNLS8 mice are also available Charles River Laboratories International Inc., Wilmington, Massachusetts, U.S.A.

### Liquid Compositions

A contemplated pharmaceutical composition of an embodiment of the present invention is typically designed for intravenous (IV) administration. Pharmaceutical composition embodiments are also contemplated for per-oral administration via tablet, capsule, and buccal, liquid compositions as solutions or dispersions, liquid compositions that are encased in a GI-tract soluble or disintegrable coating, and the like.

A typical concentration of an RB compound (RB derivative), as noted above, in a liquid formulation is about 0.1% to about 20% (w/v) using disodium rose bengal (RBD) as a basis. An amount of rose bengal itself or a rose bengal derivative, as discussed above, that is utilized in a pharmaceutical composition is an amount based on a weight amount of DSRB discussed herein such that the number of moles of a rose bengal, a salt such as DSRB or a rose bengal derivative are the same.

A contemplated liquid composition of an embodiment of the present invention is typically a liquid at room temperature, e.g., about 20 to about 25 degrees C. A principal diluent present in a liquid composition is water, which preferably constitutes at least about 90 percent by weight/volume (% w/v) of the composition, and more preferably at least about 95% w/v.

Because the cellular targets of a contemplated pharmaceutical composition of the present invention are both upper motor neurons (UMN) in the cortex and lower motor neurons (LMN) in the brainstem and spinal cord, that composition is typically intended for parenteral administration such as by subcutaneous (SC), intramuscular (IM), or intravenous (IV) methods so that the medication can be present immediately in the blood stream so that some of the “first pass” diminution of the active drug can be avoided.

Parenterally administered liquid compositions often contain an electrolyte, and preferably have approximately physiological osmolality and pH value. A preferred concentration of singly charged electrolyte ions in a pharmaceutically acceptable aqueous medium is about 0.5% to about 1.5% (w/v), more preferably at about 0.8% to about 1.2% (w/v), and most preferably at a concentration of about 0.9% (w/v). The about 0.9% (w/v) concentration is particularly preferred because it corresponds to an approximately isotonic aqueous solution. In a further preferred embodiment, the electrolyte in a contemplated liquid pharmaceutical composition is sodium chloride.

Electrolytes at such levels increase the osmolality of a pharmaceutically acceptable aqueous medium. Thus, as an alternative to specifying a range of electrolyte concentrations, osmolality can be used to characterize, in part, the electrolyte level of the composition. It is preferred that the osmolality of a composition be greater than about 100 mOsm/kg, more preferably that the osmolality of the composition be greater than about 250 mOsm/kg, and most preferably that it be about 300 to about 500 mOsm/kg. Normal saline that contains sodium chloride at 9.0 g per liter provides an osmolality of about 308 mOsm/L.

It is preferred that the pH value of a pharmaceutically acceptable aqueous medium be about 4 to about 9, to yield maximum solubility of the RB compound in an aqueous vehicle and assure compatibility with biological tissue. A particularly preferred pH value is about 5 to about 8, and more preferably between about 6 to about 7.5. At these pH values, the halogenated xanthenes typically remain in dibasic form, rather than the water-insoluble lactone that forms at low pH values.

### Solid Compositions

It is further contemplated that a pharmaceutical composition be administered in a form that is solid a room temperature for and is adapted for oral administration that is enterically-coated to pass through the stomach and release the RB compound relatively close to the site of the cancer (closer than the mouth) so that there will be a lesser amount of wasted RB compound bound to tissues dorsal to the site of the tumor and less likely visible tissue staining. The RB compound is typically dissolved in or dispersed in or on a solid diluent matrix, and the resulting solid pharmaceutical composition is a solid at room temperature.

There are several factors at play in the dissolution of an orally administered solid pharmaceutical product in a mammalian body. Among those factors are residence time of the medicament at different locations along the GI tract, particle size, solubility of the individual components of the medicament in the bodily fluids likely to be encountered from mouth to anus, the order in which various coating layers, when present, are applied to the medicament, as well as the pH value at which a particular coating layer is soluble.

For example, the highly acidic gastric environment (pH 1.5-2 in the fasted state; pH 3-6 in the fed state) rises rapidly to about pH 6 in the duodenum and increases along the small intestine to pH 7.4 at the terminal ileum. The pH value in the cecum drops just below pH 6 and again rises in the colon reaching pH 6.7 at the rectum [Hua, Front Pharmacol 11: Article 524 (April 2020)]. Observation of solutions of disodium RB mixed into a water solution having the pH value of the human stomach revealed rapid clouding of the admixture and clumping of the previously soluble disodium RB, presumably into the lactone form.

Gastric transit can range from 0 to 2 hours in the fasted state and can be prolonged up to 6 hours in the fed state. In general, the transit time in the small intestine is considered relatively constant at about 3 to about 4 hours, but can range from about 2 to about 6 hours in healthy individuals. Colonic transit times can be highly variable, with ranges from 6 to 70 hours reported [Hua, Front Pharmacol 11: Article 524 (April 2020)].

One approach useful for predictable release of a medicament to a particular location in the GI tract relies upon pH-specific coatings and matrices that are dissolve or disintegrate at preselected GI tract pH values such as those noted previously. Particularly preferred for release in or near the colon, neutral or slightly alkaline pH values are utilized to release the drug in the distal part of the small intestine or in the colon.

The table below shows some examples of pH-dependent polymer coatings that have been used for the purpose of colonic targeting (local treatment) either alone or in combination, including some methacrylic resins (commercially available from Evonik Industries, AG, Essen, Germany as Eudragit®), and hydroxypropyl methylcellulose (HPMC; available from DuPont, Wilmington, DE as Methocel™; and Ashland, Inc., as Benecel™ (Wilmington, DE) derivatives. In addition to triggering release at a specific pH value range, the enteric coating can protect the incorporated active agent against the harsh GI tract environment (e.g., gastric juice, bile acid, and microbial degradation) and can create an extended and delayed drug release profile to enhance therapeutic efficiency.

The Table below lists several commercially available enteric coating polymers and the “published pH release” value from their manufacturer. The “published pH release” values are not absolute for all compositions or environments, and pH values for dissolution or disintegration stated herein are based on those published values.

pH-Dependent Polymer Coatings*

| | Published |
|---|---|
| Polymer | pH Release |
| Eudragit ® S-100 | 7.0 |
| Eudragit ® ES-30D | 7.0 |
| Eudragit ® L-100 | 6.0 |
| Cellulose acetate phthalate | 6.0 |
| Cellulose acetate trimellitate | 5.5 |
| Eudragit ® L-30D-55 | 5.5 |
| Eudragit ® L-100-55 | 5.5 |
| Hydroxypropyl methylcellulose phthalate 55 | 5.5 |
| Hydroxypropyl methylcellulose phthalate 50 | 5.0 |
| Polyvilyl acetate phthalate | 5.0 |

*[Hua, Front Pharmacol 11: Article 524 (April 2020)]

Particularly suitable (meth)acrylate copolymers include about 10% to about 30% by weight methyl methacrylate, about 50% to about 70% by weight methyl acrylate and about 5% to about 15% by weight methacrylic acid (Eudragit® FS type). Similarly suitable, are (meth)acrylate copolymers of about 20% to about 40% by weight methacrylic acid and about 80% to about 60% by weight methyl methacrylate (Eudragit® s type). Use of the word “(meth)acrylate” is used to mean that either or both of acrylate and methacrylate monomers can be used.

These coating polymers permit little if any RB compound release prior to the particles leaving the stomach. The pH value of the fluid within the duodenum typically is about 6 and rises to about 7.4 toward the ileum.

A usual tablet or lozenge can be prepared by admixture of lactose (20%) and active ingredient (80%; RB compound) mixed in a high-speed mixer (DIOSNA type P10, Osnabruck, Germany). An aqueous solution containing the excipient polyvinylpyrrolidone (PVP) such as povidone (Sigma-Aldrich International GmbH, Buchs, CH) is added in small amounts until a homogeneous composition is obtained. The moist powder mixture is screened. Tablets are subsequently made therefrom as is well-known, and dried.

The resulting tablets or lozenges are thereafter preferably coated with a protective polymer film, often using fluidized bed equipment. Film-forming polymers are normally mixed with plasticizers and release agents by a suitable process well known to skilled workers. The film formers can in this case be in the form of a solution or suspension. The excipients for the film formation can likewise be dissolved or suspended. Organic or aqueous solvents or dispersants can be used. Stabilizers can be used in addition to stabilize the dispersion (for example: Tween® 80 or other suitable emulsifiers or stabilizers).

Examples of release agents are glycerol monostearate or other suitable fatty acid derivatives, silicic acid derivatives or talc. Examples of plasticizers include propylene glycol, phthalates, polyethylene glycols, sebacates or citrates, and other substances mentioned above and in the literature.

Another preferred type of medicament is a water-soluble capsule or blister containing a plurality of particles of an RB compound such as rose bengal disodium or rose bengal lactone that is covered with one or more layers of polymeric resin that release the RB compound quickly upon dissolution or disintegration of the capsule in water or body fluid. Capsules are typically made of gelatin and are often referred to as gelcaps. Gelatin is an animal product. Vegetarian capsules are often made of hydroxypropyl methyl cellulose (HPMC).

In some other embodiments, the RB compound is directly layered with one or more coats of the polymer to form particles that are generally spherical in shape. Such particles are often referred to as beads. In a preferred aspect, particles (beads) are sized so as that about 90 percent by weight pass through a 20 mesh sieve (opening=850 μm) screen and about 90% by weight are retained on an 80 mesh sieve (opening=180 μm) screen.

Exemplary pH value-sensitive coating polymeric resins are discussed above. Exemplary pH value-insensitive coating polymeric resins are discussed above. The pH value-sensitivity of coating polymeric resins is to be understood in terms of physiologically present pH values along the GI tract such as those discussed above.

In further embodiments, small pellets such as sugar/starch seeds, nonpareils or prills, which are small, generally spherically-shaped cores, are coated with one or a plurality of layers of the RB compound and one or more layers of polymeric coating. Illustrative sugar/starch cores are sugar spheres NF that pass through an about 40 mesh sieve (425 mm opening) screen to an about 50 mesh sieve (300 mm opening) screen, that contain not less than 62.5% and not more than 91.5% sucrose, calculated on the dry basis, the remainder consisting primarily of starch. (USP NF 1995 2313).

In an illustrative example, a 100-kilogram (kg) quantity of disodium rose bengal, a 7.1 kg quantity of cross-linked carboxymethyl cellulose (preferably croscarmellose sodium NF), and an 11.9 kg quantity of starch NF, are each divided in half, and the three constituents are blended together to form two identical batches. Each of the batches is milled through an 80 mesh screen using a mill such as a Fitzpatrick Mill. The two milled batches are then blended to form a mixture, which is tested for composition in accordance with accepted quality assurance testing methods that are well-known by those skilled in the art.

The disodium rose bengal mixture is subsequently divided into three equal parts, with a first part remaining whole, and second and third parts each divided into lots of 50%, 30%, and 20%. A 25.6 kg quantity of 40-50 mesh sugar/starch seeds, (e.g., sugar spheres NF) is placed in a stainless-steel coating pan. An 80-liter (L) quantity of 5 percent povidone/IPA solution is prepared for spraying onto the particles.

The coating pan is started with the sugar spheres, onto which is sprayed an application (approximately 0.173 kg per application) of the povidone-alcohol solution, and onto which is sifted an application (approximately 0.32 kg) of the disodium rose bengal mixture from the first part (that part that remained whole). Sifting is done using a standard sifter. The spraying and sifting steps are continued until the first part of the mixture has been applied to the sugar spheres to form a batch of partially coated spheres.

The partially coated spheres are then divided into two equal lots, each lot being placed in a coating pan. Separately for each of the two lots, spraying of the povidone/IPA solution and sifting of the disodium rose bengal mixture as divided into the 50% lots continues until the 50 percent lots have been applied to the spheres. Following application of the 50% lots, the spheres can be screened using a 25 mesh screen if necessary.

The spraying of the povidone/IPA solution and sifting of the disodium rose bengal mixture as divided into the 30 percent lots commences and continues until the 30 percent lots have been applied to the spheres. The coated spheres can be rescreened using a 25 mesh screen.

Spraying of the povidone/IPA solution and sifting of the disodium rose bengal mixture continues using the mixture as divided into the 20 percent lots until the 20% lots have been applied to the spheres. At this point in the process, the entire quantity of the disodium rose bengal mixture has been applied to the spheres, and about 50 kg of the 5% povidone/IPA solution has been applied to the spheres.

A 7.5 percent povidone/IPA solution is prepared and applied to the spheres as a sealant. The sealed spheres are tumble dried for about one hour, weighed, and placed in an oven at about 122° F. (50° C.) for 24 hours. After drying, the spheres are screened through a 20 mesh screen and a 38 mesh screen to form the immediate (quick or fast as compared to delayed) release particles.

The above-discussed RB compound-containing spheres or their capsule (or blister) can also be coated with a pH value-sensitive enteric coating polymer as discussed previously so that once released in the GI tract, the spheres do not provide their active ingredient, RB compound, to their surroundings unless the pH value is that of a desired GI tract location.

Another way to control the location of RB compound release is to further coat the spheres (RB-coated particles) discussed above, with a dissolution-controlling coat of polymeric resin applied to the surface of the spheres such that the release of the RB compound from the spheres is controlled and released over an about 6- to about 10-hour period. The materials used for this purpose can be, but are not limited to, ethylcellulose, hydroxypropylmethylcellulose, hydroxypropylcellulose, methylcellulose, hydroxyethylcellulose, nitrocellulose, or carboxymethylcellulose, as well as copolymers of acrylic acid and methacrylic acid (Eudragit®), or any other acrylic acid derivative (Carbopol®, etc.) can be used.

In addition, an enteric coating material can also be employed, either singularly, or in combination to the above non-pH-sensitive coatings. These materials include, but are not limited to, hydroxypropylmethylcellulose phthalate and the phthalate esters of all the cellulose ethers. In addition, phthalate esters of the acrylic acid derivatives (Eudragit®), or cellulose acetate phthalate.

These coating materials can be employed in coating the surfaces in an amount of about 1.0% (W/W) to about 25% (W/W). Preferably, these coating materials are present at about 8.0 to about 12.0 percent (W/W).

### Excipients

Excipients customary in pharmacy can be employed in a manner known per se in the production of the drug form. These excipients can be present in the core or in the coating agent.

### Polymers

Polymeric materials used as adhesives in helping to adhere an RB compound to a sugar pill or sphere is deemed to be an excipient where coating layers of an RB compound are employed. Illustrative of such polymers are polyvinyl pyrrolidone and polyvinyl alcohol as are other water-soluble, pharmaceutically acceptable film-forming polymers such as hydroxypropyl cellulose.

### Dryers (Non-Stick Agents)

Dryers have the following properties: they have large specific surface areas, are chemically inert, are free-flowing and comprise fine particles. Because of these properties, they reduce the tack of polymers containing polar comonomers as functional groups. Examples of dryers are: alumina, magnesium oxide, kaolin, talc, fumed silica, barium sulphate, and cellulose.

### Disintegrants

Disintegrants are added to oral solid dosage forms to aid in their disaggregation. Disintegrant are formulated to cause a rapid break-up of solids dosage forms on contacting moisture. Disintegration is typically viewed as the first step in the dissolution process. Illustrative disintegrants include sodium croscarmellose, an internally cross-linked sodium carboxymethyl cellulose, cross-linked polyvinylpyrrolidone (crospovidone) and sodium starch glycolate.

### Release Agents

Examples of release agents are: esters of fatty acids or fatty amides, aliphatic, long-chain carboxylic acids, fatty alcohols and their esters, montan waxes or paraffin waxes and metal soaps; particular mention should be made of glycerol monostearate, stearyl alcohol, glycerol behenic acid ester, cetyl alcohol, palmitic acid, carnauba wax, beeswax, and the like. The usual proportionate amounts are in the range from 0.05% by weight to 5%, preferably 0.1% to 3% by weight based on the copolymer.

### Other Excipients Customary in Pharmacy

Mention should be made here of, for example, stabilizers, colorants, antioxidants, wetting agents, pigments, gloss agents. They are typically used as processing aids and are intended to ensure a reliable and reproducible production process and good long-term storage stability. Further excipients customary in pharmacy may be present in amounts from 0.001% by weight to 10% by weight, preferably 0.1% to 10% by weight, based on the polymer coating.

### Plasticizers

Substances suitable as plasticizers ordinarily have a molecular weight between 100 and 20,000 and comprise one or more hydrophilic groups in the molecule, e.g. hydroxyl, ester or amino groups. Citrates, phthalates, sebacates, castor oil are suitable. Examples of further suitable plasticizers are alkyl citrates, glycerol esters, alkyl phthalates, alkyl sebacates, sucrose esters, sorbitan esters, dibutyl sebacate, and polyethylene glycols 4000 to 20 000. Preferred plasticizers are tributyl citrate, triethyl citrate, acetyl triethyl citrate, dibutyl sebacate, and diethyl sebacate. The amounts used are between 1 and 35, preferably 2% to 10% by weight, based on the (meth)acrylate copolymer.

Each of the patents, patent applications and articles cited herein is incorporated by reference. The articles “a” and “an” are used herein to refer to one or to more than one (i.e., to at least one) of the grammatical object of the article.

The foregoing description and the examples are intended as illustrative and are not to be taken as limiting. Still other variations within the spirit and scope of this invention are possible and will readily present themselves to those skilled in the art.

1. A method for treating a subject diagnosed with amyotrophic lateral sclerosis (ALS) that comprises administering to said subject a pharmaceutical composition containing dissolved or dispersed therein a SOD1 and/or TDP-43 aggregation-inhibiting amount of a rose bengal (RB) compound, a pharmaceutically acceptable salt of RB, rose bengal lactone, a RB amide whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic RB derivative, wherein the aromatic derivative is an ester or amide formed from an alcohol or monosubstituted amine having a 5- or 6-membered aromatic ring, or a 5,6- or 6,6-fused aromatic ring system that contains 0, 1, or 2 hetero ring atoms that are independently nitrogen, oxygen or sulfur.

2. The method according to claim 1, wherein said aromatic ring substituent is selected from one or more of the group consisting of one or more of 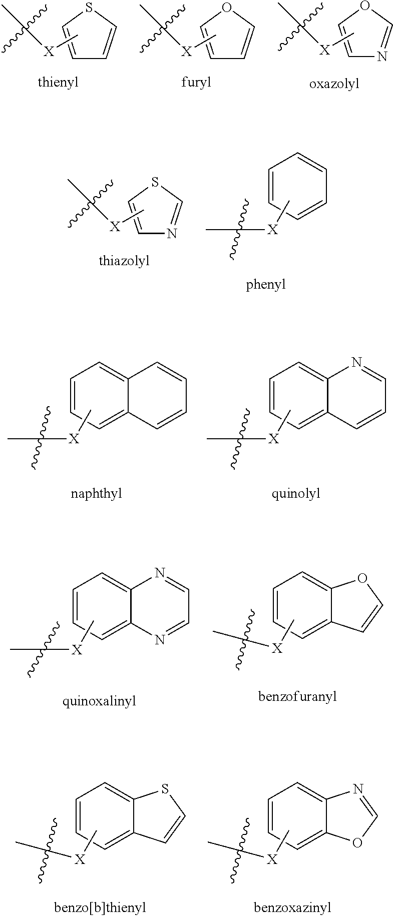
where 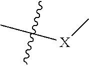
is 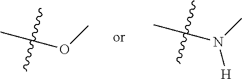
providing an ester, amide (—NH₂) or a monosubstituted amide, respectively.

3. The method according to claim 1, wherein said RB compound is a C₁-C₄ alkyl ester or rose bengal disodium salt.

4. The method according to claim 3, wherein said RB compound is rose bengal disodium salt.

5. The method according to claim 1, wherein said pharmaceutical composition is a liquid at room temperature.

6. The method according to claim 5, wherein liquid pharmaceutical composition is an aqueous composition containing at least about 90% w/v water.

7. The method according to claim 6, wherein said aqueous liquid pharmaceutical composition has an osmolality of about 300 to about 500 mOsm/kg.

8. The method according to claim 1, wherein said pharmaceutical composition is a solid at room temperature.

9. The method according to claim 1, wherein said pharmaceutical composition contains a SOD1 aggregation-inhibiting amount of said RB compound.

10. The method according to claim 1, wherein said pharmaceutical composition contains a TDP-43 aggregation-inhibiting amount of said RB compound.

11. The method according to claim 1, wherein the subject is a human.

12. The method according to claim 1, wherein the method is repeated as prescribed a plurality of times over the following months or years or until the subject is no longer in need.

13. A pharmaceutical composition for treating a subject diagnosed with amyotrophic lateral sclerosis (ALS), the pharmaceutical composition containing dissolved or dispersed therein a SOD1 and/or TDP-43 aggregation-inhibiting amount of a rose bengal (RB) compound, a pharmaceutically acceptable salt of RB, rose bengal lactone, a RB amide whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic RB derivative, wherein the aromatic derivative is an ester or amide formed from an alcohol or monosubstituted amine having a 5- or 6-membered aromatic ring, or a 5,6- or 6,6-fused aromatic ring system that contains 0, 1, or 2 hetero ring atoms that are independently nitrogen, oxygen or sulfur.

14. The pharmaceutical composition according to claim 13, wherein the subject is a human.

15. The pharmaceutical composition according to claim 13, wherein said aromatic ring substituent is selected from one or more of the group consisting of one or more of 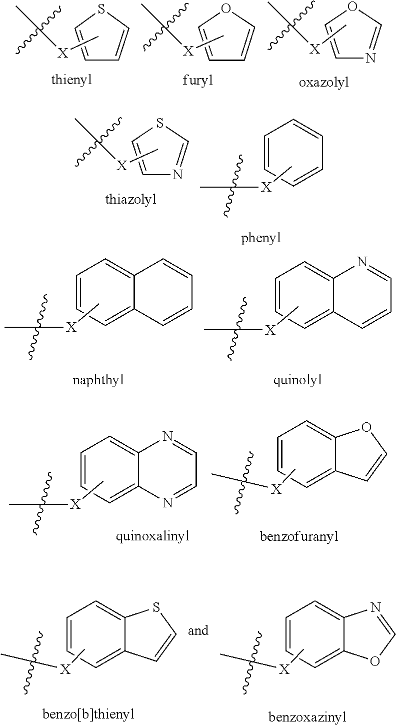
where 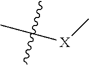
is 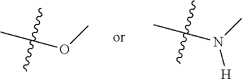
providing an ester, where is amide (—NH₂) or a monosubstituted amide, respectively.

16. The pharmaceutical composition according to claim 13, wherein said RB compound is a C₁-C₄ alkyl ester or rose bengal disodium salt.

17. The pharmaceutical composition according to claim 16, wherein said RB compound is rose bengal disodium salt.

18. The pharmaceutical composition according to claim 13, wherein said pharmaceutical composition is a liquid at room temperature.

19. The pharmaceutical composition according to claim 18, wherein liquid pharmaceutical composition is an aqueous composition containing at least about 90% w/v water.

20. The pharmaceutical composition according to claim 19, wherein said aqueous liquid pharmaceutical composition has an osmolality of about 300 to about 500 mOsm/kg.

21. The pharmaceutical composition according to claim 13, wherein said pharmaceutical composition is a solid at room temperature.

22. The pharmaceutical composition according to claim 13, wherein said pharmaceutical composition contains a SOD1 aggregation-inhibiting amount of said RB compound.

23. The pharmaceutical composition according to claim 13, wherein said pharmaceutical composition contains a TDP-43 aggregation-inhibiting amount of said RB compound.
